(19) United States
(12) Patent Application Publication
(10) Pub. No.: US 2011/0130457 A1
(43) Pub. Date: Jun. 2, 2011

(54) Wax-Based Emulsion for the Treatment of Dry Eye Conditions

(75) Inventors: Douglas Borchman, Louisville, KY (US); Gary N. Foulks, Louisville, KY (US)

(73) Assignee: UNIVERSITY OF LOUISVILLE RESEARCH FOUNDATION, INC., Louisville, KY (US)

(21) Appl. No.: 13/057,694

(22) PCT Filed: Aug. 6, 2009
(86) PCT No.: PCT/US09/52966

§ 371 Date: Feb. 4, 2011

Related U.S. Application Data
(60) Provisional application No. 61/086,482, filed on Aug. 6, 2008.

(51) Int. Cl. A61K 31/22; A61P 27/04

(52) U.S. Cl. 514/549

(57) ABSTRACT

Disclosed herein is a wax-based emulsion for the treatment of dry eye conditions by reducing the evaporation rate of saline and increasing the viscosity of the tear film. The composition of the present invention comprises an emulsion of wax dispersed in saline. One embodiment of the wax-based emulsion of the present invention comprises a wax in an amount ranging from about 0.001% to about 80% by weight and the remainder saline. In another embodiment, the wax-based emulsion further comprises a hydrocarbon in an amount ranging from about 0.01% to about 80% by weight. In still another embodiment, the wax-based emulsion of the present invention further comprises a cholesterol ester ranging in an amount from about 0.01% to about 80% by weight. In an alternative embodiment, the wax-based emulsion of the present invention further comprises an opthalmic drug in an amount ranging from about 0.01% to about 80% by weight. In yet another embodiment, the wax based emulsion comprises a wax in an amount ranging from about 40% to about 60% by weight, a cholesterol ester in an amount ranging from about 10% to about 20% by weight and the remainder saline. The wax based emulsion may further comprise a hydrocarbon in an amount of up to about 20% by weight. In yet another embodiment, the emulsion further comprises an amount ranging between about 1% to about 20% by weight of standard opthalmic drugs such as antibiotics for delivery to the eye. In some embodiments, the wax of the emulsion is a synthetic wax.

### CROSS-REFERENCE TO RELATED APPLICATIONS

This application claims the benefit of U.S. Provisional Application 61/086,482 which was filed on Aug. 6, 2008 and is hereby incorporated by reference.

### STATEMENT REGARDING FEDERALLY SPONSORED RESEARCH OR DEVELOPMENT

This invention was made with Government support under Grant No. EYO17094-01, awarded by the National Institutes of Health (NIH). The Government has certain rights in this invention.

### REFERENCE TO A “MICROFICHE APPENDIX”

Not applicable

### BACKGROUND OF THE INVENTION

Normally, the eye is covered with a multilayered tear film which protects the surface of the eye keeping it moist, lubricated and comfortable. The tear film is composed of a combination of protein, water, and oils. The system which is responsible for producing the tear film is called the lacrimal functional unit and includes the lacrimal gland, meibomian glands and goblet cells of the conjunctiva. The lacrimal glands produce the watery portion of the tear film called the aqueous. The aqueous contains certain proteins. The meibomian glands produce the lipids that are in the outermost layer of the tear film. The lipid layer prevents loss of the aqueous layer due to evaporation.

Clinically, patients may express a combination of two forms of dry eye disease, aqueous deficient and evaporative. A thorough review of the types of dry eye disease may be found in Lemp M. A. Epidemiology and classification of dry eye, Adv. Exp. Med. Bio. 1998; 438:791-803 and Lemp M., Baudouin C., Baum J., et al, The Definition and Classification of Dry Eye Disease: Report of the Definition and Classification Subcommittee of the International Dry Eye Workshop, The Ocul. Surf. 2007; 5:75-92. Both mechanisms of development of dry eye share the common feature of instability of the tear film with rapid tear breakup time, suggesting there may be a shared structural abnormality of the tear film that is responsible for the instability. Tear film break-up and dry spot formation is the sum of three processes: evaporation, tangential flow, and inward flow of tears across the surface of the cornea. Fluid flow into or out of the cornea was an insignificant factor for tear film breakup according to Nichols J. J., Mitchel G. L., King-Smith E. P., Thinning rate of the precorneal and prelens tear film, Invest Ophthalmol. Vis. Sci. 2005; 46:2353-2361. It has been estimated that 36% of tears are lost to evaporation by W. Mathers. (Mathers W., Evaporation from the ocular surface, Exp. Eye Res. 2004; 78:389-394), however, it has also been suggested that evaporation is too slow to explain completely the tear film breakup.

Rates of evaporation for normal human tears have been published in at least 18 papers (see Table 1) and range from 0.0011 to 1.9 μm/min. The data published prior to 2003 was discussed and reviewed by Mathers. For comparison, the rate of evaporation for water at 24° C. is about 10 μm/min at 20% relative humidity and 5 μm/min near a relative humidity of 60%. Temperature, humidity, and wind velocity significantly affect the rate of evaporation of water and human tears.

Evaporation rates for water and human tears are discussed in detail in Hisatake K, Tanaka S, Aizawa Y., Evaporation rate of water in a vesse, J. Appl. Physics 1993; 11:7395-7401, Tsubota K, Yamada M., Tear evaporation from the ocular surface, Invest Ophthalmol Vis Sci. 1992; 33:2942-2950, and Uchiyama E., Aronowicz J D, Butovich I A, et al., Increased evaporative rates in laboratory testing conditions simulating airplane cabin relative humidity: an important factor for dry eye, Eye Contact Lens. 2007; 33:174-176.

TABLE 1
Evaporation Rates Measured in vivo.

| Evaporation | Relative | |
|---|---|---|
| rate | Humidity | |
| (μm/min)^{a} | (%) | Reference |
| 3.8 | 49 | Nichols J. J., Mitchel G. L., King-Smith E. P., Thinning rate of |
| | | the precorneal and prelens tear film, Invest Ophthalmol Vis Sci. |
| | | 2005; 46: 2353-2361. |
| 1.9 | 40 | Yamada M, Tsubota K., Measurement of tear evaporation from |
| | | ocular surface, Nippon Ganka Gakkai Zasshi-Acta Societatis |
| | | Ophthalmologicae Japonicae 1990; 94: 1061-1070. |
| 1.6 | | Hamano H., The change of precorneal tear film by the |
| | | application of contact lenses (Japanese). Contact Intraocular |
| | | Lens Med J. 1981; 7: 205-209; |
| | | Hamano H, Hori M, Kawabe H, et al., Modification of the |
| | | superficial layer of the tear film by the secretion of the |
| | | meibomian glands, Folia Ophthalmol. Japonica 1980; 31: 353-360. |
| 1.2 | | Tomlinson A, Trees G. R., Occhipinti J. R., Tear production and |
| | | evaporation in the normal eye, Ophthal. Physiol. Optics. |
| | | 1991; 11: 44-47. |
| 0.91 | 30 | Mathers W. D., Lane J. A., Sutphin J. E., et al. Model for ocular |
| | | tear film function, Cornea. 1996; 15: 110-119. |
| 0.89 | 30 | Mathers W. D., Ocular evaporation in meibomian gland |
| | | dysfunction and dry eye, Ophthalmology. 1993; 100: 347-351. |
| 0.88 | 30 | Mathers W. D., Binarao G., Petroll M., Ocular water evaporation |
| | | and the dry eye. A new measuring device, Cornea. |
| | | 1993; 12: 335-340. |
| 0.78 | 30 | Mathers W. D., Daley T. E., Tear flow and evaporation in patients |
| | | with and without dry eye, Ophthalmology. 1996; 103: 664-669. |
| 0.73 | 40 | Mathers W. D., Ocular evaporation in meibomian gland |
| | | dysfunction and dry eye, Ophthalmology. 1993; 100: 347-351. |
| 0.65 | 22 | Uchiyama E., Aronowicz J. D., Butovich I. A., et al, Increased |
| | | evaporative rates in laboratory testing conditions simulating |
| | | airplane cabin relative humidity: an important factor for dry eye, |
| | | Eye Contact Lens. 2007; 33: 174-176. |
| 0.54 | 40 | Tsubota K., Yamada M., Tear evaporation from the ocular |
| | | surface, Invest Ophthalmol. Vis. Sci. 1992; 33: 2942-2950. |
| 0.53 | 30 | McCulley J. P., Aronowicz J. D., Uchiyama E., et al, |
| | | Correlations in a change in aqueous tear evaporation with a |
| | | change in relative humidity and the impact, Am J Ophthalmol. |
| | | 2006; 141: 758-760. |
| 0.46 | 30-40 | Shimazaki J., Sakata M., Tsubota K., Ocular surface changes |
| | | and discomfort in patients with meibomian gland dysfunction, |
| | | Arch Ophthalmol. 1995; 113: 1266-1270. |
| 0.37 | 42 | Uchiyama E., Aronowicz J. D., Butovich I. A., et al, Increased |
| | | evaporative rates in laboratory testing conditions simulating |
| | | airplane cabin relative humidity: an important factor for dry eye, |
| | | Eye Contact Lens. 2007; 33: 174-176. |
| 0.36 | 40 | McCulley J. P., Aronowicz J. D., Uchiyama E., et al, |
| | | Correlations in a change in aqueous tear evaporation with a |
| | | change in relative humidity and the impact, Am J Ophthalmol. |
| | | 2006; 141: 758-760. |
| 0.28 | | Liu D. T., Di Pascuale M. A., Sawai J., et al, Tear film dynamics |
| | | in floppy eyelid syndrome, Invest Ophthtalmol Vis Sci. |
| | | 2005; 46: 1188-1194. |
| 0.25 | 12 | Goto E., Endo K., Suzuki A., et al, Tear evaporation dynamics |
| | | in normal subjects and subjects with obstructive meibomian |
| | | gland dysfunction, Invest Ophthalmol Vis Sci. 2003; 44: 533-539. |
| 0.25 | | Matsumoto Y., Dogru M., Goto E., Tsubota K., Increased tear |
| | | evaporation in a patient with ectrodactyly-ectodermal dysplasia- |
| | | clefting syndrome, Jpn. J. Ophthalmol. 2004; 48: 372-375. |
| 0.24 | | Rolando M., Refojo M. F., Tear evaporimeter for measuring |
| | | water evaporation rate from the tear film under controlled |
| | | conditions in humans, Exp. Eye Res. 1983; 36: 25-33. |
| 0.024 | 50 | Craig J. P., Tomlinson A., Importance of the lipid layer in human |
| | | tear film stability and evaporation, Optom. Vis. Sci. 1997; 74: 8-13. |
| 0.0012 | 50 | Craig J. P., Singh I., Tomlinson A., The role of tear physiology in |
| | | ocular surface temperature, Eye. 2000; 14: 635-641. |

The evaporation rates measured for human tears in vivo (see Table 1) are much lower than about 10 μm/min, the value expected for water at 34° C. and 30% relative humidity. The difference between rates of evaporation of water, measured in vitro, and tears, measured in vivo, has been used to estimate the contribution of the tear film lipid to reduce the rate of evaporation by Mathers W. D., Lane J. A., Sutphin J. E., et al, Model for ocular tear film function, Cornea. 1996; 15:110-119. Rather than comparing the evaporation rate of tears measured in vivo with water, a better comparison would be to compare the rates of tears measured in vivo with the rate of tears measured in vitro since unlike water, tears contain salts, proteins and lipids associated with proteins which could affect the evaporation rate.

Changes in the protein concentration of the tear film with age and dry eye disease are unlikely to directly affect the rate of evaporation of tears. With the lipid layer intact, the high reserve capacity of the lacrimal gland to provide both un-stimulated and stimulated tear flow is more than enough to compensate for evaporative loss. However, with dry eye the lipid layer is not intact leading to increased rates of evaporation and decreased lacrimal tear flow which contribute to tear film break up time. Lipid volume of the tear film and the rate of evaporation in patients with dry eye are correlated. (See Mathers W., Evaporation from the ocular surface, Exp. Eye Res. 2004; 78:389-394; Shimazaki J., Sakata M., Tsubota K., Ocular surface changes and discomfort in patients with meibomian gland dysfunction, Arch. Ophthalmol. 1995; 113:1266-1270; Yokoi N., Mossa F., Tiffany J. M., et al, Assessment of meibomian gland function in dry eye using meibometry, Ophthalmology. 1999; 117:723-729; Mathers W. D., Daley T. E., Tear flow and evaporation in patients with and without dry eye, Ophthalmology. 1996; 103:664-669; Goto E., Endo K., Suzuki A., et al, Tear evaporation dynamics in normal subjects and subjects with obstructive meibomian gland dysfunction, Invest Ophthalmol. Vis. Sci. 2003; 44:533-539.)

Many patients suffering from Dry Eye or Chronic Dry Eye use over-the-counter lubricants or artificial tears, but these products offer short term or no relief from the condition. Various lipid emulsion eye drops are also available on the market. These products increase the lipid layer thickness in addition to adding to the aqueous layer as opposed to the artificial tears and lubricants which attempt to replenish the aqueous layer. For example, Soothe® is a meta-stable oil-in-water emulsion which contains Restoryl,® a light mineral oil, and a dual surfactant system intended for the relief of dry eye symptoms. (See Scaffidi, Robert C. and Donald R. Korb, Comparison of the Efficacy of Two Lipid Emulsion Eyedrops in Increasing Tear Film Lipid Layer Thickness, Eyes & Contact Lens, Vol. 33, No. 1: 38-41 (2007).) The active ingredients in Soothe® are light mineral oil (1.0%) and mineral oil (4.5%). Patients complain about discomfort and blurring after using oil-water emulsions according to Khanal, Santosh, et al., Effect of an Oil-in-Water Emulsion on the Tear Physiology of Patients With Mild to Moderate Dry Eye, Cornea, Vol. 26, No. 2: 175-181 (Feb. 2007). The discomfort and blurring are attributable to the non-natural substances used in the emulsion, namely oil. This discomfort may cause excessive blinking resulting in expelling the emulsion from the eye shortly after it is added. Additionally, as the name implies, these emulsions are not stable and may separate upon standing for several hours. Before these types of emulsions can be added to the eye, they must be shaken to reform the emulsion. (See U.S. Pat. No. 5,371,108, col. 7, 11. 57-62.) This reformation by shaking is not as precise as the original formation and; therefore, can result in poor mixing and a poorly formed emulsion.

Refresh® Dry Eye Therapy is another lipid emulsion intended to slow the evaporation of the tear film. The active ingredients in Refresh® are glycerin (1%) and polysorbate 80 (1%). While this product increases the viscosity of the tear film, it does not necessarily increase the lipid layer. Studies have shown that any increase of the lipid layer after addition of the Refresh® type product to the eye, dissipates after 15 minutes. Any decrease in the evaporation rate due to this product is due to the increased viscosity of the aqueous layer, not any increase in the lipid layer.

U.S. Pat. No. 5,371,108 (“Korb Patent”) discloses a meta-stable oil and water emulsion which is applied to the eye in a gel form. The majority of the solution is comprised of oil. Oil is not a natural lipid in the human meibum, a layer of the tear film. Oil forms globules on the surface of the eye and does not decrease the rate of evaporation. Oil also contributes to the instability of the emulsion. Upon standing, meta-stable oil and water emulsions like the one disclosed in the Korb Patent separate into an oil phase and a water phase. The biphasic mixture is not suitable for application to the eye. A small amount of wax is dissolved in the oil of the Korb Patent; however, only naturally occurring waxes are utilized in the Korb Patent. These naturally occurring waxes are complex mixtures which contain triglyceride impurities. These tryglyceride impurities are broken down via a high temperature alkali hydrolysis treatment, but the treatment introduces 10% to 20% by weight of free fatty alcohols, another impurity, into the wax. These alcohols are not naturally found in the human meibum and could bind to and change the structure of the proteins in tears thereby deranging the native conformational structure of the native tear lipid layer. Additionally, the Korb Patent utilizes phospholipids which are either completely absent from the native meibum, or present at negligible amounts not exceeding 0.5%. Phospholipids are subject to oxidation resulting in deleterious secondary products of lipid oxidation.

### BRIEF SUMMARY OF THE INVENTION

Applicants' invention relates to the eye lubricants which reduce the evaporation rate of the tear film and increases the tear film viscosity. More specifically, Applicants' invention discloses a wax based emulsion utilizing ingredients that are naturally present in the tear layer in the eye that replenishes and thickens the lipid layer of the eye's tear film thus reducing the evaporation rate of the aqueous layer and minimizing the effects of dry eye conditions. The present invention also increases the viscosity of the tear film thereby increasing tear film break-up time. The composition of the present invention comprises an emulsion of wax dispersed in saline. One embodiment of the present invention comprises a wax based emulsion for the treatment of dry eye conditions comprising a wax in an amount ranging from about 0.001% to about 80% by weight and the remainder saline. In another embodiment, the wax based emulsion comprises a wax in an amount ranging from about 0.001% to about 80% by weight, a hydrocarbon in an amount ranging from about 0.01% to about 80% by weight and the remainder saline. In still another embodiment, the wax based emulsion comprises a wax in an amount ranging from about 0.001% to about 80% by weight, a cholesterol ester in an amount ranging from about 0.01% to about 80% by weight and the remainder saline. The wax based emulsion may further comprise a hydrocarbon in an amount ranging from about 0.01% to about 80%. In yet another embodiment, the emulsion further comprises an amount ranging between about 0.01% to about 80% by weight of standard opthalmic drugs for delivery to the eye.

Still another embodiment of Applicants' invention comprises a wax based emulsion for the treatment of dry eye conditions comprising a wax in an amount ranging from about 40% to about 60% by weight and the remainder saline. In another embodiment, the wax based emulsion comprises a wax in an amount ranging from about 40% to about 60% by weight, a hydrocarbon in an amount of up to about 20% by weight and the remainder saline. In yet another embodiment, the wax based emulsion comprises a wax in an amount ranging from about 40% to about 60% by weight, a cholesterol ester in an amount ranging from about 10% to about 20% by weight and the remainder saline. The wax based emulsion may further comprise a hydrocarbon in an amount of up to about 20% by weight. In yet another embodiment, the emulsion further comprises an amount ranging between about 1% to about 20% by weight of standard opthalmic drugs such as antibiotics for delivery to the eye.

In one embodiment, the wax is a synthetic wax monoester such as palmityloleate, palmityl palmitate, stearyl palmitate, myristyl dodecanoate, and arachidyl dodecanoate.

The wax based emulsion of the present invention provides a direct method of treating dry eye conditions by replenishing the lipid layer of the tear film thereby preventing evaporation of the aqueous layer and improving dry eye conditions. It also provides a vehicle for delivery of opthalmic drugs to the eye. Unlike other lipid emulsions for the treatment of dry eye conditions, the wax based emulsion of the present invention is stable for long periods of time. The wax based emulsion of the present invention utilizes ingredients that are natural to the eye and does not require preservatives. The wax based emulsion may include a cholesterol ester to improve the cholesterol ester deficiency experienced by many patients with dry eye disease. The cholesterol ester also increases the viscosity of the emulsion thereby reducing its rate of evaporation. Due to the hydrophobic nature of the wax, hydrophobic drugs can be mixed in the emulsion and added to the eye.

### BRIEF DESCRIPTION OF THE DRAWINGS

FIG. 1 is a graphical illustration of the evaporation rates for phosphate buffered saline in still room air (relative humidity 40.6%) and dry moving air at 25° C. and 34° C.

FIG. 2 is a graphical illustration of the evaporation rate for emulsions with different lactoglobulin concentrations as reported in Table 2.

FIG. 3 is a graphical illustration of the evaporation rate as compared to the thickness of the palmityloleate layer added to the surface of the protein mixture.

FIG. 4 is a graphical illustration of the tryptophan fluorescence intensity as compared to the thickness of the palmityloleate layer added to the surface of the protein mixture.

### DETAILED DESCRIPTION OF THE INVENTION

The present invention comprises a wax based emulsion for the treatment of dry eye conditions comprising wax dispersed in saline. Wax is the primary lipid utilized in the emulsion of the present invention. Wax is also the primary lipid in the human meibum (about 80%). The wax should be similar to the waxes found in the tear layer in the eye. The wax chosen should have an order to disorder phase transition temperature that is plus or minus 20° Celsius of the phase transition temperature of native meibum which is 29° Celsius. Those of skill in the art will recognize that many types of waxes could be utilized in the emulsion. Wax esters that cover a wide range of molecular weights and saturation such as those suggested for cholesterol esters and hydrocarbons are suitable for the present invention. For example, wax monoesters such as palmityloleate, palmityl palmitate, stearyl palmitate, myristyl dodecanoate, and arachidyl dodecanoate are used in some embodiments. Natural waxes such as cabana wax and beeswax are also suitable for use in other embodiments of the present invention. In one embodiment, waxes with 12 to 24 carbon chains lengths are utilized.

Waxes similar to those found in tears that are synthetically made are preferred because they have minimal impurities and are more stable. Synthetic waxes such as such as palmityloleate, palmityl palmitate, stearyl palmitate, myristyl dodecanoate, and arachidyl dodecanoate are used in one embodiment of the present invention. When synthetic waxes are used, there is no need to remove any impurities from these synthetic waxes using methods such as high temperature alkaline treatment. Such treatments, while removing some undesirable impurities from the wax, often leave other impurities in the treated wax which are not natural to the eye. For example, natural waxes often contain triglycerides which must be broken down into free fatty alcohols using high temperature alkali hydrolysis. These free fatty alcohols, which are not native to human meibum, comprise about 10% to 20% by weight of the hydrolyzed wax and could bind to and change the structure of proteins in tears thereby deranging the native conformational structure of the native tear lipid layer. Additionally, the wax chosen should bind to the natural tear proteins in the tear film thereby allowing the wax to spread over the surface of the eye without the addition of unnatural surfactants to the emulsion. Such surfactants may derange the native conformational structure of the lipid on the surface of the tear layer.

Any saline solution could be utilized in the wax-saline emulsion, for example, phosphate buffered saline without calcium or magnesium with a pH of 7.2 or a 60% balanced salt solution. In one embodiment an amount ranging between about 0.001% and about 80% by weight of wax is dispersed saline solution. In another embodiment a wax in an amount ranging between about 40% and about 60% by weight is dispersed in a 60% balanced salt solution. In yet another embodiment, the wax is a synthetic wax monoester. In another embodiment, the wax is palmityloleate and the saline solution is phosphate buffered saline solution.

In some embodiments, a small amount of hydrocarbon is added to the wax and saline emulsion. For example, in one embodiment, the present invention comprises an mixture of between about 0.001% and about 80% by weight of wax and between about 0.01% and about 80% by weight hydrocarbon dispersed in saline. In still another embodiment, the present invention comprises an mixture of between about 40% and about 60% by weight of wax and up to about 20% by weight of hydrocarbon dispersed in saline. The hydrocarbon selected should be similar to the hydrocarbons found in the tear film such as n-tetradecane, n-hexane, n-hexadecane, n-decane, n-octacosane, 9-hexadecane, 5,8,11,14-icosatetraenane, 3-methyltetredecane, and 5-propyloctacosane. Those of skill in the art will recognize that many hydrocarbons can be used in the present invention. In one embodiment, hydrocarbons with carbon chain lengths of 6 to 24 carbons are selected. Saturated hydrocarbons are preferred because polyunsaturated hydrocarbons are unstable and oxidize readily. The hydrocarbon selected should be pure and free of the secondary products of lipid oxidation. Products of lipid oxidation such as malondialdehyde may also irritate the eye and cause damage.

In an alternative embodiments, cholesterol ester is added to the wax and saline emulsion. For example, in one embodiment, the invention comprises a mixture of between about 0.001% and about 80% of wax and a cholesterol ester in an amount ranging between about 0.01% and about 80% by weight dispersed in saline. In another embodiment, hydrocarbon in an amount ranging between about 0.01% and about 80% by weight is added to the wax, ester, and saline solution. In still another embodiment, the present invention comprises a mixture of between about 40% and about 60% by weight of wax and between about 10% and about 20% by weight cholesterol ester dispersed in saline. In yet another embodiment, hydrocarbon in an amount of up to about 20% by weight is added to the wax, ester, and saline solution.

Cholesterol esterfied to fatty acids covering a wide range of molecular weights and saturation is used. The cholesterol ester should be similar to cholesterol esters found in the tear film such as cholesterol oleate, hexanate, palmitate, oleate, arachidate, and montanate. Those of skill in the art will recognize that there are many suitable cholesterol esters that may be added to the emulsion. In one embodiment, cholesterol esters have 6 to 24 carbon chain fatty acids. Applicants believe that the addition of a cholesterol ester to the emulsion of the present invention will have the added benefit of replacing the cholesterol ester in the tear film. The cholesterol ester level in the tear film has been found to be low in patients with meibomian gland dysfunction (dry eye disease). Cholesterol esters increase the order or stiffness of the hydrocarbon chains found in waxes and hydrocarbons; therefore, Applicants believe that the addition of cholesterol ester will reduce the rate of evaporation of the aqueous layer of the tear film. Additionally, Applicants believe that the presence of a cholesterol ester will increase the viscosity of the emulsion of the present invention, which in turn, will reduce the evaporation rate of the tear film.

Further, low surface tension and high surface pressure is important to the spreading of tears. De-lipidated tears have a high surface tension which can be lowered by adding meibum lipid, which is comprised of mostly wax and cholesterol esters, back to the tear layer. A high concentration of mucin alone (about 10 mg/ml) lowers the surface tension of tears. It is believed that cholesterol esters increase the surface pressure of the meibum causing it to spread.

In yet another embodiment, standard opthalmic drugs are added to the emulsion for delivery to the eye. Those of skill in the art will recognize that many opthalmic drugs can be added to the emulsion of the present invention for delivery to the eye. For example, opthalmic drugs such as antibiotics and lipid soluble drugs may be added to the emulsion. Due to the hydrophobic nature of the wax, Applicants believe that hydrophobic drugs can be added to the emulsion for delivery to the eye where they are incorporated in the tear film. It is unexpected that the hydrophobic wax would mix well with saline. The ability to add hydrophobic drugs to the eye using the wax based emulsion as a vehicle as an unexpected improvement. In one embodiment of the present invention, an opthalmic drug in an amount ranging between about 0.01% and about 80% by weight are mixed with the emulsion for delivery to the eye. In yet another embodiment, the emulsion further comprises an amount ranging between about 1% to about 20% by weight of an opthalmic drug for delivery to the eye.

The wax based emulsion of the present invention is prepared by mixing the wax with saline solution. In embodiments where hydrocarbon, cholesterol ester and/or opthalmic drugs are included in the composition, those compounds are combined with the wax and saline. Those of skill in the art will recognize that there are many ways to prepare the wax based emulsion of the present invention. One method that is used for preparing the emulsion of the present invention is described below. The compounds are mixed with a vortex mixer, such as a Genie-2 mixer (ThermoFisher Scientific), until blended (approximately one minute). The mixture is then sonicated using a probe sonicator, such as a microprobe sonicator (Branson Ultra Sonics Co.), for three minutes. The mixture is allowed to rest for 10 minutes. The sonication and rest steps are repeated ten times for a total sonication period of 30 minutes.

The wax based emulsion of the present invention is more stable than the emulsions that currently exist for the treatment of dry eye conditions. No preservatives are required to maintain stability. Many of the current emulsions must be re-sonicated or shaken immediately before use to re-emulsify the mixture. The wax based emulsion of the present invention is stable for long periods of time due to the improved mixing process. The physical changes during sonication cause the emulsion to be stable for weeks. For example, an emulsion comprising about 9 parts palmityloleate to about 1 part tetradecane was visually observed to be uniformly cloudy after 4 weeks of undisturbed storage. It is unexpected that wax, which is a lipid and therefore hydrophobic, would mix well with saline. For the same reason, it is unexpected that the wax-based emulsion of the present invention would remain stable for long periods of time. Due to the sonification step which disperses the lipid into a stable emulsion, wax can be used as the primary lipid in the emulsion disclosed herein. Wax is also the primary lipid (about 80%) in human meibum. It is believed that the wax of the emulsion disclosed herein would readily mix with the native wax of the meibum.

The wax based emulsion of the present invention has been shown to reduce the evaporation rate of saline when a lipid layer is layered on a simulated tear film. Applicants believe that, when the emulsion of the present invention is added to the human eye, the wax will segregate with the lipid layer of the eye's tear film thus repairing the lipid layer on the eye's surface. Applicants believe this will decrease the evaporation rate of the aqueous layer of the tear film and provide relief to patients with dry eye disease and chronic dry eye disease. The wax based emulsion of the present invention has been shown to bind to proteins similar to those found in the tear film of the eye. Applicants believe that, when the emulsion is added to the eye, some of the wax will bind to the proteins found in the tear film, thereby increasing the viscosity of the tear film. The patient's tear film break-up time will increase due to the increased viscosity of the tear film. Patients with meibomian gland dysfunction, or dry eye disease, often suffer from a lower tear film break-up time.

### EXAMPLES

Other features of the present invention will become apparent in the course of the following examples which are given for illustration of the invention and are not intended to be limiting thereof.

Experimentation was conducting using the following components. Mucin, lysozyme and lactoglobulin were used as models for tear proteins. Lactoglobulin was used as a model for lipocalin. These proteins were combined to simulate the tear film. A wax based emulsion was prepared. Palmityloleate was selected as the wax and n-tetradecane was selected as the hydrocarbon. Phosphate buffered saline without calcium or magnesium chloride and having a pH of 7.2 was selected as the saline. The phosphate buffered saline replaces the aqueous layer of the tear film in a patient with dry eye disease. Reflex human tears were obtained by stimulating a human subject.

Samples (0.750 mL) for evaporation rate measurements were placed into a plastic container 0.8 cm deep and 1.500 cm inside diameter. Temperature and relative humidity were recorded at the start and end of each experiment. Samples were weighed every minute for 10 minutes using a Mettler-Toledo AT261 analytical balance (Columbus, Ohio). The balance was calibrated and certified by a Mettler technician prior to use. The weights of as many as 7 samples were measured sequentially, i.e., after measuring the weights of sample 1 for ten minutes, the weights of sample 2 were measured for ten minutes, followed by the next sample. The weights of four 10 minute weighing sequences were plotted versus time and the rate of evaporation was calculated from the slope of the data in the plots that were fitted using least squares linear regression analysis. The rate of evaporation at 34° C. was measured using four, 3-minute weighing sequences instead of ten.

The rate of evaporation with 20 standard cubic feet per minute (SCFM) of dry air flowing over the sample at 24° C. was measured. Air was completely dried using a Keaser desiccant dry, model KLDW-10S (Columbus, Ohio) attached to a Purimetrics Compressor, model 750-2 (Edina, Minn.) and a 80 gallon air receiver tank. Complete removal of water vapor from the air was confirmed using an infrared spectrometer. The sample was placed under a flow of dry air for 1 minute and removed from the dry air for two minutes and weighed in a Mettler-Toledo analytical balance. This cycle was repeated ten times. The rate of evaporation in dry air was calculated from the total rate of evaporation minus the contribution of the evaporation in the room air. Evaporation rate was expressed as μm/min, the rate of thickness lost at the surface per minute. Changes in the thickness of the tear film (˜5 μm) due to evaporation can be estimated readily by expressing the evaporation rate in μm/min.

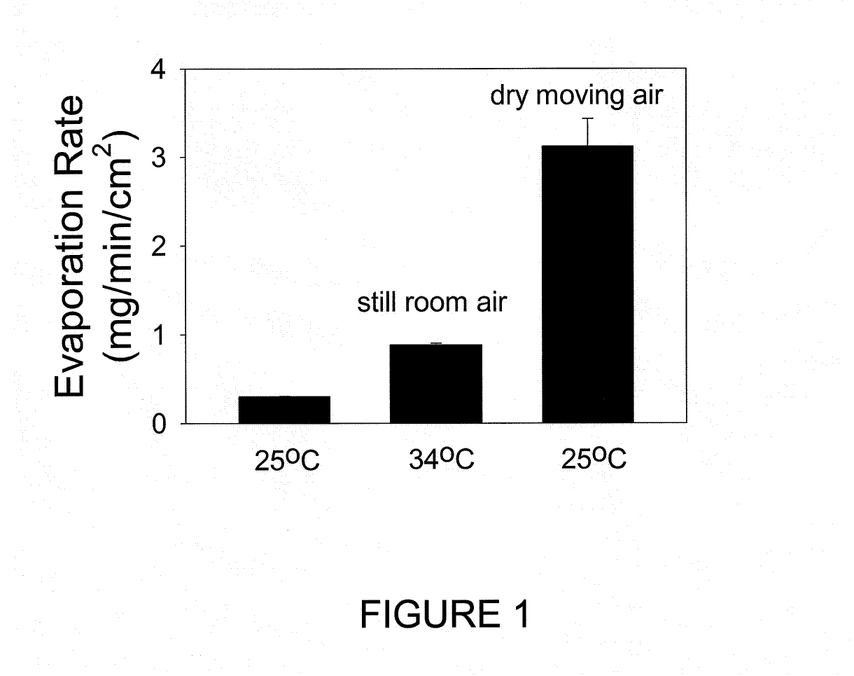

Because the temperature at the surface of the cornea can vary from 26.4° C. (ambient air temperature of −20° C.) to 36.7° C. (ambient air temperature of 40° C.), the evaporation rates of human stimulated tears and phosphate buffered saline were measured at 25° C. and 34° C. in still air and 40% humidity. Temperature significantly affects evaporation rates. As seen in Table 2, a temperature increase of 9° C. caused a threefold increase in the evaporation rates of tears and a comparable increase for the buffer. Using these data assuming and assuming no tear flow, a 5-μm-thick layer of tears on the surface of the eye would evaporate in 1.6 min (evaporation rate of 3.04 μm/min) in still air at 25° C., but it would only take 0.5 min (32 seconds, evaporation rate of 9.3 μm/min) in still air at 34° C. (FIG. 1). These rates are expected to be lower than those measured in real-world environments because dry air and wind could increase the evaporation rates. Indeed, a ten-fold increase (from 2.97 to 31 μm/min, see Table 2) was observed in the rate of evaporation of phosphate buffered saline at 25° C. when a gentle stream of dry air was applied. (See FIG. 1) Using the same example above, we estimate that at 25° C., a current of dry air could evaporate all of the tear fluid in less than 10 seconds (0.16 s) and this process would be even faster at 34° C. An intact lipid layer is essential to delay tear break-up time, especially under extreme conditions.

The comparison of evaporation rates measured in vitro in this work and the much lower values reported for in vivo measurements indicate that, although environmental factors can alter tear evaporation rates, other factors such as the presence of an intact lipid layer may contribute to reduce evaporation. The next section addresses the role of tear constituents on evaporation rates.

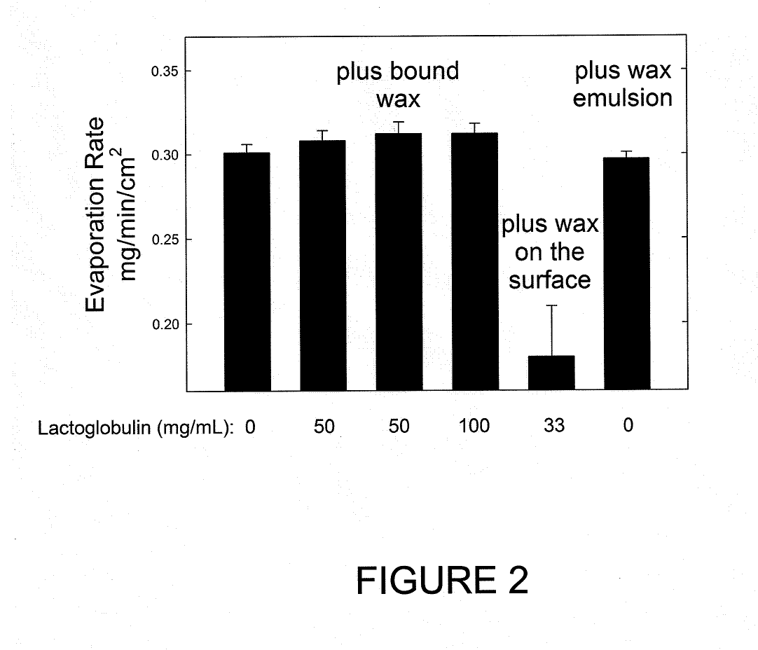

The results of the experimentation are shown in Table 2 and FIG. 2. When Table 2 indicates wax, the composition tested was an emulsion of palmityloleate and phosphate buffered saline free of calcium and magnesium. The average rate of evaporation of phosphate buffered saline was 2.97±0.04 μm/min, standard error of the mean, n=41. Evaporation rates were measured on 41 different days. The average temperature and relative humidity levels were 24.8±0.02° C. and 43±3%, respectively. The weights of the samples decreased linearly with time, with an average correlation coefficient of 0.9994. At 34° C., the evaporation rate of phosphate buffered saline was 8.0±0.5 μm/min, standard error of the mean, n=5. The rate of evaporation was 2.9 times greater at 34° C. than at 24.8° C. (FIG. 1). A similar correlation was observed for lactoglobulin solution at a concentration of 100 mg/mL in phosphate buffered saline (Table 2). A stream of dry air over phosphate buffered saline raised the evaporation rate about 10 fold to 31±3 μm/min (FIG. 1).

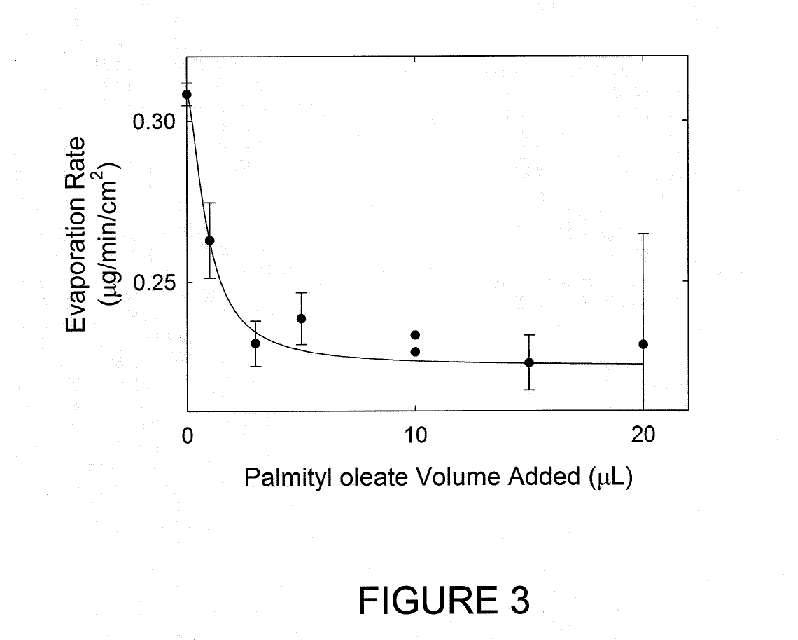

The rate of evaporation of reflex human tears was not statistically different from that for phosphate buffered saline with or without proteins as shown in FIG. 1. Evaporation rates were measured gravimetrically for phosphate buffered saline. The relative humidity of the still air samples was 40.6%. An emulsion of wax, and wax with model proteins also did not change the rate of evaporation of phosphate buffered saline. Mucin, lysozyme, lactoglobulin or wax at concentrations of 50 to 100 mg/mL did not significantly influence the rate of evaporation at 25 or 34° C. (Table 2). In contrast, a mixture of wax and hydrocarbon (1:1, wt:wt) layered on the surface of a mixture of lysozyme, mucin, and lactoglobulin decreased the rate of evaporation by 23% (Table 2). The rate of evaporation decreased with increasing amounts of lipid layered on the surface, but reached a plateau at 3 μL. Further additions of lipid up to 10 fold did not change the rate of evaporation (FIG. 3). Assuming an even distribution of lipid and based on the surface area of the sample containers, 3 μL of lipid would form a 17 μm thick layer. Applicants believe that, when the emulsion of the present invention is added to the eye, the wax will segregate with the lipid layer of the eye's tear film thus repairing the lipid layer on the surface of the tear film. Applicants believe that the effect of the addition of the emulsion to the eye will be a reduction in the evaporation rate of the aqueous layer of the tear film, similar to the results found in this experiment.

Changes in protein concentration alone are unlikely to affect the rate of evaporation of tears. This is in agreement with Pandit et al who reported surface tension results indicating that that the contribution of individual proteins to overall viscosity is small. (See Pandit J. C., Nagyova B., Bron A. J., et al, Physical properties of stimulated and unstimulated tears. Exp Eye Res. 1999; 68:247-253.) In this embodiment of the present invention, wax interacted with lactoglobulin. Wax-lactoglobulin binding was quantified by measuring changes in the intrinsic fluorescence intensity of tryptophan. Tryptophan fluorescence is sensitive to local changes in electrostatic fields. Therefore, variations in the local environment/polarity of tryptophan leads to changes in its fluorescence properties as described by Vivian and Callis. Vivian J. T., Callis P. R., Mechanisms of tryptophan fluorescence shifts in proteins, Biophys. J. 2001; 80:2093-2109.) Substrate binding and protein structural changes are commonly measured by observing changes in tryptophan fluorescence intensity, wavelength maximum, band shape, anisotropy, fluorescence lifetimes, and energy transfer.

A wax emulsion that is stable for at least 4 weeks was prepared as described previously by the inventors. (Borchman D., Foulks G. N., Yappert M. C., et al. Spectroscopic Evaluation of Human Tear Lipids, Chem Phys Lipids. 2007; 147:87-102.) Palmityloleate wax was mixed at 0.1 mg/mL with phosphate buffered saline. The sample was sonicated in an ultrasonic bath (Branson 1510, Branson Ultrasonics Co., Danbury, Conn.) for 15 min and mixed vigorously with a vortex Genie-2 mixer (ThermoFisher Scientific, Waltham, Mass.). A quantified amount of wax (0.156 to 1.25 μL/mL) was mixed with 1 mg/mL lactoglobulin in phosphate buffered saline and equilibrated for 12 hours at 34° C. under an atmosphere of argon. An ISS PC1 photon-counting spectrofluorometer (Champagne, Ill.) with a polarization accessory unit was used. Emission spectra were measured from 300 to 400 nm with an excitation wavelength of 270 nm. The peak height of the tryptophan band near 330 nm was calculated after subtracting the baseline. Steady-state fluorescence anisotropy, r, was calculated by equation 1.

r=(I_{II}−gI_{⊥})/(I_{II}+2gI_{⊥}) Equation 1

Where g=I_{⊥}/I_{II}; I_{II} and I_{⊥} are defined as the intensities of the parallel and perpendicular polarized components of fluorescence intensity relative to the plane of polarization of the excitation beam, respectively. Total fluorescence intensity (Tₜₒₜ) was calculated as:

Iₜₒₜ=I_{II}+2I_{⊥} Equation 2

All values are presented as the average±the standard error of the mean (SEM). Significance was assessed using the Student's t test for paired averages.

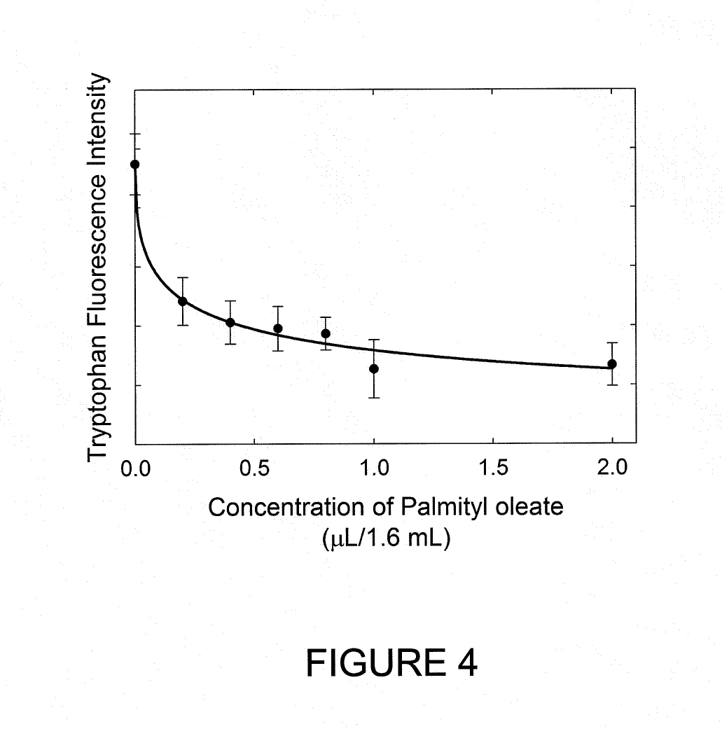

The results of this experiment are shown in FIG. 4. As little as 20 nmoles (˜0.1 μg) of palmityloleate wax added to 1 mg of β-lactoglobulin (˜10 nmoles) in 1 ml phosphate buffered saline caused a decrease in the fluorescence intensity of the tryptophan residues in lactoglobulin. Tryptophan anisotropy decreased concomitantly, p=0.04, from 0.138±0.009, n=9, with no wax to 0.115±0.004, n=6 with the addition of wax. The decrease in anisotropy and fluorescence suggests that wax, when bound to tear lipocalin, will induce a structural change in the protein and increase the viscosity of human tears. This correlation is explained in Gouveia S. M., Tiffany J. M., Human tear viscosity: An interactive role for proteins and lipids, Biochim. Biophys. Acta. 2005; 1753:155-163.

An intact lipid layer is essential to delay tear film break-up time, especially under extreme conditions. With the lipid layer intact and inhibiting the evaporation rate by 90% as suggested, all of the tears would evaporate in 30 seconds rather than 3 seconds. With the lipid layer intact, the high reserve capacity of the lacrimal gland to provide both un-stimulated and stimulated tear flow is more than enough to compensate for evaporative loss. However, with dry eye, increased rates of evaporation and decreased lacrimal tear flow result in decreased tear film break-up times. Applicants believe that, when the wax-based emulsion of the present invention is added to the eye, some of the wax will bind to the proteins found in the tear film thereby inducing a structural change in the protein which increases its viscosity and the tear film break-up time.

TABLE 2
Evaporation Rates measured in vitro.

| | | | Standard | | (p) |
|---|---|---|---|---|---|
| | | Rate of | Error of | Number | vs PBS |
| | Concentration | Evaporation | the | of | unless |
| Sample | (mg/mL) | (μm/min) | Mean | trials | indicated^{a} |
| Phosphate buffered saline | | 2.97 | 0.04 | 41 | |
| Phosphate buffered saline, 34° C. | | 8.0 | 0.5 | 3 | *0.0000 |
| Phosphate buffered saline, dry | | 31 | 3 | 4 | *0.0000 |
| moving air | | | | | |
| Human stimulated tears | | 3.04 | 0.05 | 5 | 0.30 |
| Human stimulated tears, 34° C. | | 9.3 | 0.9 | 2 | *0.0000^{b} |
| β-Lactoglobulin | 50 | 3.08 | 0.06 | 7 | 0.15 |
| β-Lactoglobulin | 100 | 3.12 | 0.06 | 6 | 0.06 |
| β-Lactoglobulin plus wax | 50:50 | 3.12 | 0.07 | 5 | 0.11 |
| β-Lactoglobulin, 34° C. | 100 | 9.1 | 0.6 | 5 | 0.26^{c} |
| Mucin | 50 | 2.9 | 0.1 | 4 | 0.55 |
| Mucin | 100 | 3.0 | 0.2 | 4 | 0.89 |
| Mucin plus wax emulsion | 50:50 | 2.98 | 0.08 | 4 | 0.92 |
| Lysozyme | 50 | 3.0 | 0.2 | 5 | 0.89 |
| Lysozyme | 100 | 3.0 | 0.1 | 7 | 0.79 |
| Lysozyme plus wax emulsion | 50:50 | 3.0 | 0.1 | 6 | 0.79 |
| Wax emulsion | 50 | 2.97 | 0.07 | 5 | 1.00 |
| Wax emulsion | 100 | 2.97 | 0.05 | 5 | 1.00 |
| Wax emulsion, 34° C. | 50 | 8.1 | 0.5 | 3 | 0.89^{c} |
| Lysozyme plus Mucin | 50:50 | 3.13 | 0.06 | 5 | 0.06 |
| Lysozyme plus Lactoglobulin | 50:50 | 3.1 | 0.2 | 4 | 0.57 |
| Lactoglobulin plus Mucin | 50:50 | 3.19 | 0.02 | 3 | *0.0000 |
| Lysozyme, Lactoglobulin, Mucin | 33:33:33 | 3.09 | 0.04 | 21 | *0.04 |
| Lysozyme, Lactoglobulin, Mucin | 33:33:33/3-20 μL | 2.289 | 0.02 | 23 | *0.0000^{d} |
| plus wax/hydrocarbon (1:1 v:v) | on top | | | | |

The temperature was 24.8° C. and the relative humidity was 40.6% unless indicated.
*statistically different p < 0.05
^{a}p was calculated using the Student's t test for unequal variances unless indicated.
^{b}p calculated by comparison with human tears at 25° C. using the Student's t test for equal variances.
^{c}p calculated by comparison with PBS at 34° C. using the Student's t test for equal variances.
^{d}p calculated by comparison with Lysozyme, Lactoglobulin, Mucin data in the row above, using the Student's t test for equal variances.
^{e}the density of water was assumed to be 1 g/cm³.

Thus, it is seen that the wax based emulsion of the present invention readily achieves the ends and advantages mentioned as well as those inherent therein. While certain preferred embodiments of the invention have been illustrated and described for the purposes of the present disclosure, numerous changes in the arrangement and construction of parts may be made by those skilled in the art, which changes are encompassed within the scope and spirit of the present invention as defined by the following claims.

What is claimed is:

1. A wax-based composition for the treatment of dry eye conditions wherein said composition is in the form of an emulsion comprising:
wax in an amount ranging from about 40% to about 60% by weight; and
the remainder saline.

2. The composition of claim 1 further comprising:
hydrocarbon in an amount of up to about 20% by weight.

3. The composition of claim 1 further comprising:
opthalmic antibiotic for delivery to the eye in an amount ranging from about 1% to about 20% by weight.

4. The composition of claim 1 further comprising:
cholesterol ester in an amount ranging from about 10% to about 20% by weight.

5. The composition of claim 1 wherein:
said wax is a synthetic wax monoester.

6. The composition of claim 1 wherein:
said wax is selected from the group consisting of palmityloleate, palmityl palmitate, stearyl palmitate, myristyl dodecanoate, and arachidyl dodecanoate.

7. The composition of claim 1 wherein:
said hydrocarbon is a saturated hydrocarbon with between 6 and 24 carbon atoms.

8. The composition of claim 1 wherein:
said opthalmic antibiotic is hydrophobic.

9. A wax-based composition for the treatment of dry eye conditions wherein said composition is in the form of an emulsion comprising:
synthetic wax in an amount of about 30% by weight;
hydrocarbon with between 6 and 24 carbon atoms in an amount of about 10% by weight;
cholesterol ester in an amount of about 10% by weight for increasing the viscosity and lowering the surface tension of the emulsion; and
the remainder saline.

10. The composition of claim 9 further comprising:
hydrophobic opthalmic antibiotic for delivery to the eye in an amount ranging from about 1% to about 20% by weight.

11. The composition of claim 9 wherein:
said wax is selected from the group consisting of palmityloleate, palmityl palmitate, stearyl palmitate, myristyl dodecanoate, and arachidyl dodecanoate; and
said hydrocarbon is an alkane isomer selected from the group consisting of decanes, hexanes, and octacosanes.

12. A wax-based composition for the treatment of dry eye conditions wherein said composition is in the form of an emulsion comprising:
wax in an amount ranging from 0.001% to 80% by weight; and
the remainder saline.

13. The composition of claim 12 further comprising:
hydrocarbon with between 6 and 24 carbon atoms in an amount ranging from 0.01% to 80% by weight.

14. The composition of claim 12 further comprising
opthalmic antibiotic in an amount ranging from 0.01% to 80% by weight.

15. The composition of claim 12 further comprising
cholesterol ester in an amount ranging from 0.01% to 80% by weight.

16. The composition of claim 12 wherein:
said wax is a synthetic wax monoester.

17. The composition of claim 12 wherein:
said wax is palmityloleate; and
said saline is phosphate buffered saline.

18. The composition of claim 13 wherein:
said hydrocarbon is n-tetradecane.

19. The composition of claim 1, wherein:
said wax and said saline are combined until blended; and
said blended mixture is then sonicated for 30 minutes in alternating periods of three minutes of sonication and 10 minutes of rest thereby inducing a physical change in the emulsion which increases stability.

20. The composition of claim 21, wherein said blended mixture further comprises:
hydrocarbon in an amount of up to about 20% by weight.

21. The composition of claim 21, wherein said blended mixture further comprises:
cholesterol ester in an amount ranging from about 10% to about 20% by weight.

22. The composition of claim 21, wherein said blended mixture further comprises:
opthalmic antibiotic for delivery to the eye in an amount ranging from about 1% to about 20% by weight.
